(19) United States
(12) Patent Application Publication
(10) Pub. No.: US 2020/0390148 A1
(43) Pub. Date: Dec. 17, 2020

(54) DEODORIZING DEVICE FOR EXHALED SMOKE OR VAPOR

(71) Applicant: KEITH HARRINGTON, Ogden (SU)

(72) Inventor: KEITH HARRINGTON, Ogden (SU)

(21) Appl. No.: 16/901,617

(22) Filed: Jun. 15, 2020

Related U.S. Application Data
(60) Provisional application No. 62/861,177, filed on Jun. 13, 2019.

(51) Int. Cl. A24F 13/00; A24F 40/51; A24F 40/48; A61L 9/12

(52) U.S. Cl. CPC A24F 13/00; A24F 40/51; A61L 2209/133; A61L 9/122; A61L 2209/212; A24F 40/48

(57) ABSTRACT

Apparatuses and methods are disclosed for deodorizing exhaled smoke or vapor. A tube may be provided for receiving a stream of exhaled air. An emitter coupled to the tube may be operable to emit a deodorizer chemical into the stream of exhaled air in the tube. A deodorizer chemical source may be coupled to the emitter for delivering the deodorizer chemical to the emitter.

### CROSS-REFERENCES TO RELATED APPLICATIONS

This application claims the benefit of U.S. Provisional Patent Application No. 62/861,177 entitled “DEODORIZING DEVICE FOR EXHALED SMOKE OR VAPOR” and filed on Jun. 13, 2019 for Keith Harrington, which is incorporated herein by reference.

### FIELD

This invention relates to odor prevention and more particularly relates to a deodorizing device for exhaled smoke or vapor.

### BACKGROUND

Smoking involves burning a substance like tobacco to inhale the smoke. Similarly, vaping involves heating or atomizing a substance without combustion, to inhale a vapor or aerosol. In either case, the exhaled smoke, vapor, or aerosol may release odors into the smoker's or vaper's surroundings. Odors produced by smoking or vaping may linger in curtains or upholstery, or may be unpleasant to others.

### SUMMARY

Apparatuses are disclosed for deodorizing exhaled smoke or vapor. An apparatus, in one embodiment, includes a tube for receiving a stream of exhaled air. In a further embodiment, an emitter coupled to the tube is operable to emit a deodorizer chemical into the stream of exhaled air in the tube. In a further embodiment, a deodorizer chemical source is coupled to the emitter for delivering the deodorizer chemical to the emitter.

In one embodiment, one or more baffles are disposed in the tube to mix the deodorizer chemical with the stream of exhaled air before the stream of exhaled air exits the tube. The deodorizer chemical, in some embodiments, includes an oxidizing agent. In one embodiment, the oxidizing agent is chlorine dioxide. In another embodiment, the oxidizing agent is ozone.

In one embodiment, a mouthpiece is coupled to an intake end portion of the tube, for receiving the stream of exhaled air from a user. In a further embodiment, a fan is disposed in the tube and operable with the emitter to propel the stream of exhaled air toward an exhaust end portion of the tube. In some embodiments, an air intake opening coupled to the intake end portion permits the tube to receive a secondary airflow in addition to the stream of exhaled air.

In some embodiments, an apparatus includes a receptacle for holding an ingredient to be atomized and inhaled. In further embodiments, an apparatus includes an atomizer coupled to the receptacle and the tube, the atomizer operable to atomize the ingredient for inhalation through the mouthpiece. In one embodiment, an airflow-operated switch operates the emitter in response to a user exhaling through the mouthpiece, and operates the atomizer in response to the user inhaling through the mouthpiece. In another embodiment, a switch is operable by a user to operate the emitter. In some embodiments, a sensor disposed in the tube is configured to produce an emitter activation signal in response to sensing the stream of exhaled air. In some embodiments, control circuitry is configured to operate the emitter in response to the emitter activation signal.

In some embodiments, the deodorizer chemical source is a pressurized gas cartridge and the emitter is a valve. In some embodiments, the deodorizer chemical source is a liquid reservoir and the emitter is an atomizer. In some embodiments, the deodorizer chemical source stores the deodorizer chemical. In some embodiments, the deodorizer chemical source produces the deodorizer chemical from one or more precursor chemicals. In some embodiments, the deodorizer chemical source provides one or more precursors to the emitter, and the emitter produces the deodorizer chemical from the one or more precursor chemicals. In some embodiments, the deodorizer chemical source is a replaceable cartridge. In further embodiments, an apparatus may include a cartridge level indicator.

Methods are disclosed for deodorizing exhaled smoke or vapor. A method, in one embodiment, includes receiving a stream of exhaled air in a tube. In a further embodiment, a method includes delivering a deodorizer chemical from a deodorizer chemical source to an emitter coupled to the tube. In some embodiments, a method includes operating the emitter to release the deodorizer chemical into the stream of exhaled air in the tube.

In certain embodiments, a method includes operating a fan disposed in the tube to propel the stream of exhaled air past one or more baffles. In further embodiments, propelling the stream of exhaled air past one or more baffles may mix the deodorizer chemical with the stream of exhaled air before the stream of exhaled air exits the tube.

### BRIEF DESCRIPTION OF THE DRAWINGS

In order that the advantages of the invention will be readily understood, a more particular description of the invention briefly described above will be rendered by reference to specific embodiments that are illustrated in the appended drawings. Understanding that these drawings depict only typical embodiments of the invention and are not therefore to be considered to be limiting of its scope, the invention will be described and explained with additional specificity and detail through the use of the accompanying drawings, in which:

FIG. 1 is a side view illustrating one embodiment of an apparatus for deodorizing exhaled smoke or vapor;

FIG. 2 is a side view illustrating another embodiment of an apparatus for deodorizing exhaled smoke or vapor;

FIG. 3 is a cross section view illustrating another embodiment of an apparatus for deodorizing exhaled smoke or vapor;

FIG. 4 is a side view illustrating a deodorizer chemical source and an emitter, in one embodiment;

FIG. 5 is a side view illustrating a deodorizer chemical source and an emitter, in another embodiment;

FIG. 6 is a side view illustrating a deodorizer chemical source and an emitter, in another embodiment;

FIG. 7 is a side view illustrating a deodorizer chemical source and an emitter, in another embodiment;

FIG. 8 is a schematic flow chart diagram illustrating one embodiment of a method for deodorizing exhaled smoke or vapor; and

FIG. 9 is a schematic flow chart diagram illustrating another embodiment of a method for deodorizing exhaled smoke or vapor.

### DETAILED DESCRIPTION

Reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

Furthermore, the described features, structures, or characteristics of the invention may be combined in any suitable manner in one or more embodiments. In the following description, numerous specific details are included to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

As used herein, a list with a conjunction of “and/or” includes any single item in the list or a combination of items in the list. For example, a list of A, B and/or C includes only A, only B, only C, a combination of A and B, a combination of B and C, a combination of A and C or a combination of A, B and C. As used herein, a list using the terminology “one or more of” includes any single item in the list or a combination of items in the list. For example, one or more of A, B and C includes only A, only B, only C, a combination of A and B, a combination of B and C, a combination of A and C or a combination of A, B and C. As used herein, a list using the terminology “one of includes one and only one of any single item in the list. For example, “one of A, B and C” includes only A, only B or only C and excludes combinations of A, B and C. As used herein, “a member selected from the group consisting of A, B, and C,” includes one and only one of A, B, or C, and excludes combinations of A, B, and C.” As used herein, “a member selected from the group consisting of A, B, and C and combinations thereof” includes only A, only B, only C, a combination of A and B, a combination of B and C, a combination of A and C or a combination of A, B and C.

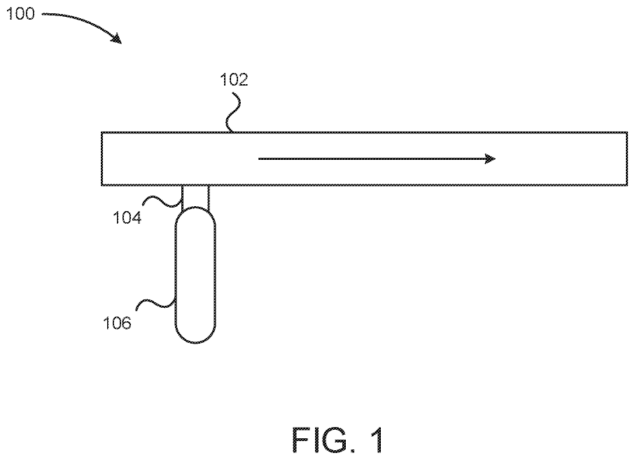

FIG. 1 depicts one embodiment of an apparatus 100 for deodorizing exhaled smoke or vapor. The apparatus 100, in the depicted embodiment, includes a tube 102, an emitter 104, and a deodorizer chemical source 106. A user may exhale smoke, vapor, or aerosol into the tube 102, and the emitter 104 may emit a deodorizer chemical from the deodorizer chemical source 106 into the tube 102. In various embodiments, exhaling smoke, vapor, or aerosol through a tube 102 to be mixed with a deodorizer chemical may result in the smoke, vapor, or aerosol being significantly deodorized before it exits the tube 102.

In the depicted embodiment, a tube 102 is provided for receiving a stream of exhaled air. The term “exhaled air” is used herein to encompass whatever a user might blow or exhale into the tube 102, which may include gases from ambient air such as oxygen or nitrogen, and may also include other components mixed with or suspended in the air such as smoke from a cigarette, cigar or pipe, atomized droplets from a vaporizer, or the like. Thus, exhaled smoke or vapor, while not typically regarded as “air,” may still be encompassed by the term “exhaled air.” Similarly, a stream of gases and other components flowing from a user's mouth into the tube 102 may be referred to as “exhaled air” regardless of whether the stream was circulated through the user's lungs or puffed into and out of the user's mouth without a deeper inhalation.

The stream of exhaled air flows through the tube 102 from an intake end portion, to an exhaust end portion. The direction of airflow for the stream of exhaled air is indicated by a right-pointing arrow in FIG. 1. Thus, in FIG. 1, the intake end portion of the tube 102 is a portion of the tube 102 toward the left of the Figure, and the exhaust end portion of the tube 102 is a portion of the tube 102 toward the right of the Figure.

The tube 102, in various embodiments, may be any structure capable of conveying a fluid flow (e.g., the stream of exhaled air) from a first end opening to a second end opening. An elongate central cavity may couple the end openings and may be surrounded (except at the end openings) by tube walls. Thus, in one embodiment, a tube 102 may be a hollow cylinder, open at both ends, with a circular cross section. In another embodiment, a tube 102 may have a non-circular cross section. For example, a tube 102 may have a cross-section that is elliptical, square, rectangular, hexagonal, or the like. Walls of the tube 102 may be formed from one or more rigid or flexible materials, in various embodiments, such as metal, plastic, composite materials, or the like. In one embodiment, the tube 102 may be formed of a single material. In another embodiment, the tube 102 may include multiple materials. For example, the interior of the tube 102 may be lined or coated with a second material, or may be passivated in a chemical reaction forming a layer of a second material, to prevent the deodorizer chemical from reacting with the walls of the tube 102.

The emitter 104, in the depicted embodiment, is coupled to the tube 102, and is operable to emit a deodorizer chemical into the stream of exhaled air in the tube 102. Smoke, vapor, or atomized droplets in the stream of exhaled air may include chemicals that cause odors. Releasing these chemicals into the smoker's or vaper's surroundings may result in odors lingering in curtains, upholstery or other areas that trap odor. Additionally, releasing odor-carrying chemicals into the smoker's or vaper's surroundings may be irritating or unpleasant to others. Accordingly, in various embodiments, a deodorizer chemical may be a chemical capable of reacting with odoriferous chemicals in the stream of exhaled air. The products of such a chemical reaction may have a reduced or changed odor.

In one embodiment, a deodorizer chemical may be an oxidizing agent that oxidizes one or more chemical components of the stream of exhaled air. For example, a deodorizer chemical may be an oxidizing agent such as chlorine dioxide, ozone, hydrogen peroxide, or the like. An oxidizing agent may break chemical bonds in odoriferous chemicals to produce chemicals with reduced or different odors. In another embodiment a deodorizer chemical may be a cyclodextrin that binds or encapsulates odoriferous chemicals. Various other or further chemicals used for deodorization may similarly be used as a deodorizer chemical in further embodiments of an apparatus 100 for deodorizing exhaled smoke or vapor.

The emitter 104, in various embodiments, may be any structure capable of emitting a deodorizer chemical into the stream of exhaled air. Emitting a chemical into the stream of exhaled air may include releasing or discharging a chemical into the tube 102, producing a chemical that flows into or through the tube 102, or the like. In one embodiment, the deodorizer chemical may be pressurized, and the emitter 104 may be a valve that, when open, permits the deodorizer chemical to expand into the tube 102. In another embodiment, the deodorizer chemical may be in the form of a liquid such as an aqueous solution, and the emitter 104 may be an atomizer that emits the liquid into the tube 102 as aerosolized droplets. An atomizer as an emitter 104 may emit aerosolized droplets by spraying a liquid deodorizer chemical through a nozzle, heating the liquid using a small heating element, vibrating the liquid at a high frequency (e.g., using an ultrasonic piezo atomizer), or the like.

In another embodiment, the emitter 104 may emit the deodorizer chemical by producing the deodorizer chemical from one or more precursor chemicals. For example, in one embodiment, the emitter 104 may be an ozone generator that produces ozone using stored oxygen or oxygen from ambient air to produce ozone and release the ozone into the tube 102. In another embodiment, the emitter 104 may provide a space for two or more precursor chemicals to mix and react, to produce a deodorizer chemical that flows into the tube 102. Various other or further devices capable of producing or releasing chemicals may be used as an emitter 104, in various embodiments.

In various embodiments, the emitter 104 is operable by a user or by electronic control circuitry. Operating the emitter 104 may include operating a mechanical lever or valve actuator, operating a switch for an electrically operated emitter 104, providing power to a heat element, providing ultrasonic-frequency voltage to a piezo atomizer, sending a control signal to an electronically controlled emitter 104, mixing precursor chemicals, or the like. In one embodiment, an emitter 104 may be manually operated by a user of the apparatus 100. For example, a user may press a button or operate a switch to operate the emitter 104. In another embodiment, an emitter 104 may be automatically operated to emit the deodorizer chemical when the stream of exhaled air is flowing through the tube 102. For example, electronic control circuitry may turn the emitter 104 on or off in response to a signal produced by an airflow sensor.

The deodorizer chemical source 106, in the depicted embodiment, is coupled to the emitter 104 for delivering the deodorizer chemical to the emitter 104. Delivering the deodorizer chemical to the emitter 104 may include making the deodorizer chemical available in any way at the emitter 104, such as by supplying a stored deodorizer chemical to the emitter 104, producing the deodorizer chemical from one or more precursor chemicals to supply the produced deodorizer chemical to the emitter 104, supplying one or more precursor chemicals to the emitter 104 for the emitter 104 to produce the deodorizer chemical, or the like.

In certain embodiments, where the deodorizer chemical is produced by the apparatus 100, either the deodorizer chemical source 106 or the emitter 104 may produce the deodorizer chemical. For example, in one embodiment with ozone as the deodorizer chemical, the deodorizer chemical source 106 may be an ozone generator, and the emitter 104 may be a valve operable to release ozone into the tube 102. In another embodiment, with ozone as the deodorizer chemical, the deodorizer chemical source 106 may be an oxygen source, such as a cartridge of pressurized oxygen gas or an opening permitting oxygen in ambient air to reach the emitter 104, and the emitter 104 may be an ozone generator. Production of other deodorizer chemicals may similarly occur at the deodorizer chemical source 106 or the emitter 104, in various embodiments. In another embodiment, production of a deodorizer chemical in a multi-step reaction may include steps at both the deodorizer chemical source 106 and the emitter 104.

In one embodiment, the deodorizer chemical source 106 stores the deodorizer chemical. For example, where the deodorizer chemical is a gas, the deodorizer chemical source 106 may be a pressurized gas cartridge, and the emitter 104 may be a valve. Similarly, where the deodorizer chemical is a liquid, the deodorizer chemical source 106 may be a liquid reservoir and the emitter 104 may be an atomizer. In an embodiment, where the deodorizer chemical is produced by the apparatus 100, the deodorizer chemical source 106 may include one or more containers, or separated areas within a larger container, to store one or more precursor chemicals. In some embodiments, the deodorizer chemical source 106 may be a replaceable cartridge, allowing a user to replenish supplies of the deodorizer chemical. In another embodiment, the apparatus 100 may be a disposable device, and the deodorizer chemical source 106 may be non-replaceable, or permanently coupled to the emitter 104.

In some embodiments, where the deodorizer chemical source 106 produces the deodorizer chemical, the deodorizer chemical source 106 may include space for reacting one or more precursor chemicals, a device for mixing or reacting one or more precursor chemicals, or the like. For example, where the deodorizer chemical is chlorine dioxide, the deodorizer chemical source 106 may include space for powdered sodium chlorite to mix with liquid water to produce chlorine dioxide. Various other or further types of containers, gas cartridges, liquid reservoirs, reaction equipment, or the like may be suitable for use as a deodorizer chemical source 106.

The deodorizer chemical source 106 may be coupled to the emitter 104 in a variety of ways. For example, the deodorizer chemical source 106 may be coupled to the emitter 104 via a threaded fitting, a fitting with a pin for piercing a gas cartridge, a quick-connect push fitting, a coupling that immerses an atomizer-based emitter 104 in a liquid deodorizer chemical, a coupling that wicks a liquid deodorizer chemical to an atomizer-based emitter 104, or the like. Various other or further types of fittings, couplings, or connectors may be used to couple the deodorizer chemical source 106 to the emitter 104.

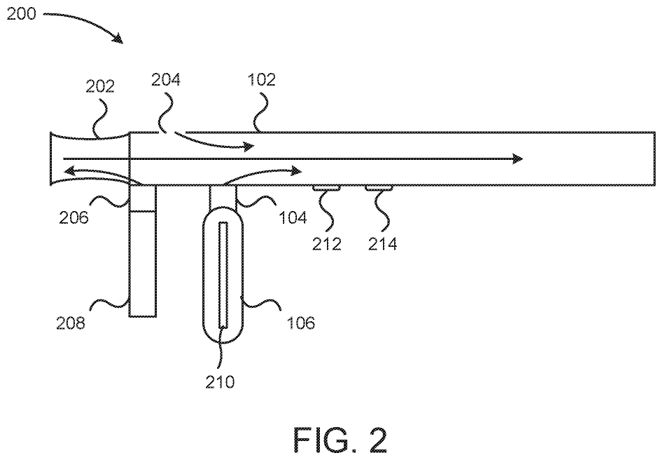

FIG. 2 depicts another embodiment of an apparatus 200 for deodorizing exhaled smoke or vapor. The apparatus 200, in the depicted embodiment, may be substantially similar to the apparatus 100 described above with reference to FIG. 1, including a tube 102, an emitter 104, and a deodorizer chemical source 106 as described above. In the depicted embodiment, the apparatus 200 includes a mouthpiece 202, an air intake opening 204, a receptacle 208 for holding an ingredient to be atomized and inhaled, an atomizer 206, a cartridge level indicator 210, and two switches 212, 214, which are described below. In certain embodiments, an apparatus for deodorizing exhaled smoke or vapor may include some of the components depicted in FIG. 2 while omitting other components. For example, a mouthpiece 202 may be included or omitted independently of whether an air intake opening 204 is included or omitted in an apparatus.

As in FIG. 1, a horizontal arrow indicates the flow of the stream of exhaled air through the tube 102 from left to right, with the intake end portion of the tube 102 on the left of the Figure and the exhaust end portion of the tube 102 on the right. Additionally, an arrow into the tube 102 from the emitter 104 shows the direction of flow for the deodorizer chemical in FIG. 2, from the emitter 104 toward the exhaust end portion at the right of the Figure.

The mouthpiece 202, in the depicted embodiment, is coupled to the intake end portion of the tube 102, for receiving the stream of exhaled air from the user. A mouthpiece 202, in various embodiments, may be a hollow structure capable of conveying the stream of exhaled air from the user's mouth to the intake end portion of the tube 102. A mouthpiece 202 may be made of various materials, such as metal, plastic, silicone, silicone-coated metal, or the like. In one embodiment, a mouthpiece 202 may be detachable from the tube 102 (e.g., for a user to sanitize). In another embodiment, a mouthpiece 202 may be non-detachably coupled to the tube 102.

A mouthpiece 202, in one embodiment, may be the same shape or cross-section as the tube 102. In another embodiment, a mouthpiece 202 may be shaped to engage a user's mouth. For example, in one embodiment, a mouthpiece 202 may be tapered, indented, rounded or the like, for comfort when a user inserts the mouthpiece 202 into his or her mouth to blow into the tube 102. In another embodiment, a mouthpiece 202 may be funnel-shaped so that the mouthpiece 202 surrounds a user's lips when the user blows into the tube 102. In certain embodiments, a mouthpiece 202 may be a commercially available mouthpiece for a vape pen or a pipe. In some embodiments, providing a shaped mouthpiece 202 may facilitate a good seal between the user's lips and the apparatus 200 so that exhaled smoke or vapor enters the apparatus 200 to be deodorized, without producing a side-stream of non-deodorized smoke or vapor that does not enter the tube 102.

In the depicted embodiment, an air intake opening 204 is coupled to the intake end portion of the tube 102 (e.g., the left portion of the tube 102 in FIG. 2, near the mouthpiece 202). An air intake opening 204 may be an opening that admits air into the tube 102. Thus, the air intake opening 204 may permit the tube 102 to receive a secondary airflow (e.g., of ambient air) in addition to the stream of exhaled air. The direction of secondary airflow is indicated by an arrow into the tube 102 from the air intake opening 204 in FIG. 2.

In some embodiments, oxidation of odoriferous chemicals in smoke or vapor may be facilitated by admitting additional air into the tube 102 in a secondary airflow. In some embodiments, a fan may propel the stream of exhaled air through the tube 102, and admitting a secondary airflow into the tube 102 may prevent the user from feeling suction that the fan might otherwise produce. In one embodiment, an air intake opening 204 may simply be an opening in the wall of the tube 102. For example, a fan may pull air in through an opening. In another embodiment, an air intake opening 204 may be an opening in a constricted section of the tube 102, to pull air into the tube 102 using the Venturi effect. Various other or further types of opening may be suitable for use as an air intake opening 204.

In FIG. 1, the apparatus 100 includes components for deodorizing exhaled air from someone who smokes or vapes. In FIG. 2, the apparatus 200 additionally includes equipment for vaping, so that a vaper can both inhale and exhale though the same mouthpiece 202. A receptacle 208 is provided for holding an ingredient to be atomized and inhaled. The receptacle 208 may have different forms in different embodiments, depending on the ingredient to be atomized. For example, where the ingredient is a propylene glycol or other solvent-based vape liquid, the receptacle 208 may be a liquid reservoir. Where the ingredient is a botanical extract in oil or wax form the receptacle 208 may be a container for the oil or wax. In another embodiment, the receptacle 208 may be a larger container that holds non-extracted botanical ingredients such as ground-up biomass from a plant, and the atomizer 206 may atomize some components (e.g., oils) from the biomass, leaving other components in the receptacle 208.

The atomizer 206, in the depicted embodiment, is coupled to the receptacle 208 and the tube 102, and is operable to atomize the ingredient in the receptacle 208 for inhalation through the mouthpiece 202. The flow of an atomized ingredient is indicated by an arrow into the mouthpiece 202 from the atomizer 206, in FIG. 2. In certain embodiments, the atomizer 206 and the receptacle 208 may be commercially available vape components with standardized fittings. In one embodiment, the receptacle 208 may be removed and refilled, or replaced. In another embodiment, the atomizer 206 and the receptacle 208 may be removed and replaced as a single unit.

In one embodiment, the atomizer 206 may be manually operated by a user. In another embodiment, the atomizer 206 may be automatically operated based on an airflow sensor or an airflow-operated switch (e.g., using “auto-draw” vape components). In the depicted embodiment, a user operates the atomizer 206 and the emitter 104 manually using switches 212, 214. The switches 212, 214 may couple a battery or another power source (not shown) to the atomizer 206 or the emitter 104 to operate the atomizer 206 or the emitter 104. Switch 212 is operable by a user to operate the atomizer 206. The user may hold the switch 212 and inhale the atomized ingredient. Switch 214 is operable by a user to operate the emitter 104. The user may hold the switch 214 while exhaling so that the deodorizer chemical is emitted into the stream of exhaled air. In various embodiments, one or more switches 212, 214 for operating the atomizer 206 and/or the emitter 104 may include a pair of pushbutton momentary switches, a single switch with three positions for standby, inhaling, and exhaling, or various other types of switches.

In the depicted embodiment, the deodorizer chemical source 106 is a removable or replaceable cartridge, permitting supplies of the deodorizer chemical to be renewed by refilling or replacing the cartridge. In further embodiments the apparatus 200 may include a cartridge level indicator 210. In various embodiments, a cartridge level indicator 210 may permit a user to determine whether the level of deodorizer chemical (or precursor) in a cartridge is low, or if the cartridge in need of replacement. For example, in one embodiment, a cartridge level indicator 210 may be a light-emitting diode coupled to a sensor and/or a controller that powers the light in response to determining, via the sensor, that the level in the cartridge is low. In the depicted embodiment, the cartridge level indicator 210 is a window in the side of the cartridge (e.g., a window made of glass, plastic, or another transparent material), allowing the user to directly observe the level of a chemical in the cartridge. In another embodiment, a cartridge level indicator 210 may be a pressure gauge for a pressurized gas cartridge. Various other or further types of sensors, gauges, or observation equipment may be used as a cartridge level indicator 210.

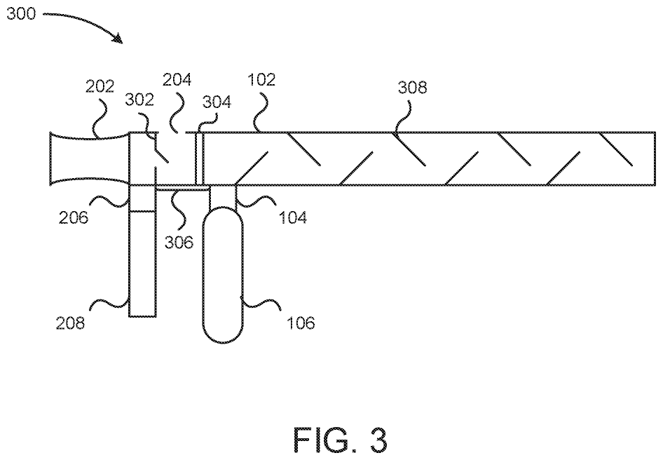

FIG. 3 depicts another embodiment of an apparatus 300 for deodorizing exhaled smoke or vapor. A cross section of the apparatus 300 is depicted to view components disposed inside the tube 102. Such components, although not shown in FIG. 1 or 2, may also be included in embodiments of the apparatuses 100, 200. The apparatus 300, in the depicted embodiment, may be substantially similar to the apparatus 100 described above with reference to FIG. 1, including a tube 102, an emitter 104, and a deodorizer chemical source 106 as described above. The apparatus 300 in the depicted embodiment includes some components described with reference to FIG. 2, such as the mouthpiece 202, air intake opening 204, atomizer 206, and receptacle 208, but omits other components such as the switches 212, 214. In the depicted embodiment, the apparatus 300 includes a sensor 302, a fan 304, control circuitry 306, and baffles 308, which are described below.

In some embodiments, an airflow-operated switch operates the emitter 104 in response to a user exhaling through the mouthpiece 202 or the tube 102, and/or operates the atomizer 206 in response to the user inhaling through the mouthpiece 202 or the tube 102. In one embodiment, a manually operated switch may operate either the atomizer 206 or the emitter 104, and an airflow-operated switch may operate the other component. In the depicted embodiment, the airflow-operated switch operates both the atomizer 206 and the emitter 104. Thus, the user may use the apparatus 300 for vaping simply by inhaling through the mouthpiece 202, and may deodorize the exhaled vapor (or aerosol) simply by exhaling through the mouthpiece 202.

In the depicted embodiment, an airflow operated switch may include a sensor 302, and may include control circuitry 306. A sensor 302 may be a device that produces an emitter activation signal in response to sensing the stream of exhaled air. In some embodiments, a sensor 302 may produce an atomizer activation signal in response to sensing inhalation. An emitter activation signal or an atomizer activation signal may be the presence or absence of a voltage or current, a particular series of voltages or currents, an “open” (non-conducting) or “closed” (conducting) switch state, or the like. For example, in the depicted embodiment, the sensor 302 includes a check valve disposed in the tube 102 between the atomizer 206 and the emitter 104. The check valve may be in a neutral position, neither fully open nor fully closed, when the user is neither inhaling nor exhaling. The check valve may close when the user inhales, and may fully open when the user exhales. The sensor 302 may include switches that close and/or open based on the position of the check valve. In another embodiment, a sensor 302 may sense inhalation or exhalation in another way, and may include an airflow sensor, a pressure sensor, or the like.

Control circuitry 306, in certain embodiments, may operate the emitter 104 in response to an emitter activation signal from the sensor 302. In a further embodiment, the control circuitry 306 may operate the atomizer 206 in response to an atomizer activation signal from the sensor 302 (or from a second sensor). In one embodiment, the sensor 302 may be switch based, and the control circuitry 306 may simply be a power source that the sensor switch(es) couple to the atomizer 206 and/or the emitter 104. In another embodiment, control circuitry 306 may include discrete logic components or circuitry, such as transistors for switching based on a signal, a timer for determining delay times (e.g., if the control circuitry 306 operates the fan 304 on a delay from the emitter 104 to allow premixing of the exhaled air and the deodorizer chemical), or the like. In another embodiment, control circuitry 306 may include a microcontroller, such as a processor executing code stored on a non-transitory computer readable medium, and the code may include instructions for the processor to communicate with the sensor 302 and operate the atomizer 206 or the emitter 104. Various other or further types of control circuitry 306 may be used in an apparatus 300.

The fan 304, in the depicted embodiment, is disposed in the tube 102, and is operable with the emitter 104. For example, the same manually-operated switch or airflow-operated switch that operates the emitter 104 may operate the fan 304. The fan 304 and the emitter 104 may be powered by the same power sources or by separate power sources. Operating the fan 304 with the emitter 104 may include operating both the fan 304 and the emitter 104 for time periods that are coterminous (so the fan 304 and the emitter 104 turn on and off together) or that are overlapping (e.g., the fan 304 may be turned on or off slightly before or after the emitter 104).

In various embodiments, a fan 304 is operable to propel the stream of exhaled air toward an exhaust end portion of the tube 102. Certain deodorizing devices for smokers or vapers may rely on filtration of the exhaled air through activated carbon or another filter medium. However, filter-based deodorizers may require a user to blow into the device with a substantial amount of pressure to force the exhaled air through the filter medium. By contrast, exhaling though the tube 102 may involve minimal air pressure from the user (to overcome air friction from baffles 308, open a check valve, or the like), and the fan 304 may facilitate airflow through the tube 102 so that the deodorizer chemical mixes with the exhaled air even if the user exhales weakly.

One or more baffles 308, in various embodiments, may be disposed in the tube 102 to mix the deodorizer chemical with the stream of exhaled air before the stream of exhaled air exits the tube 102. Baffles 308 may include any structure that promotes turbulent rather than laminar airflow in the tube 102. Turbulence may facilitate mixing of the stream of exhaled air with the deodorizer chemical. In the depicted embodiment, the baffles 308 are protrusions into the tube interior from the tube walls. In another embodiment, baffles 308 may include dimples in the tube walls, mesh in the interior of the tube 102, an auger or screw requiring air to flow in a helical path through the tube 102, or the like. Various other turbulence-producing structures may be used as baffles 308 in various embodiments.

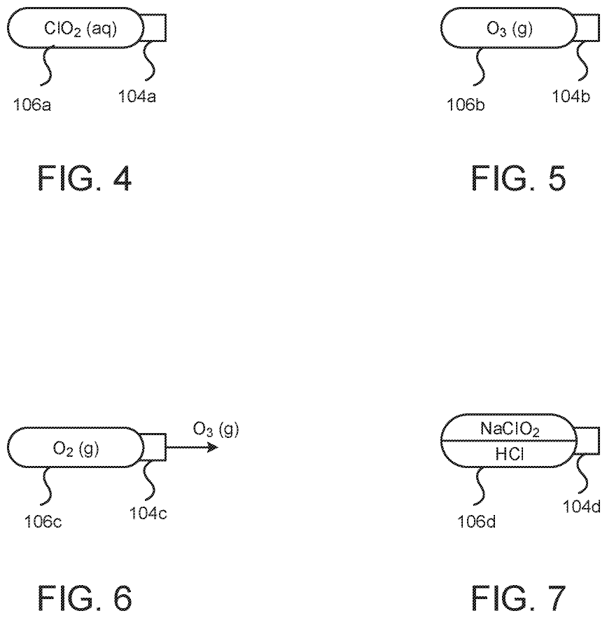

FIGS. 4-7 depict a deodorizer chemical source 106 and an emitter 104, in various embodiments. In the depicted embodiments, the deodorizer chemical sources 106a-d and the emitters 104a-d may be embodiments of the deodorizer chemical source 106 and the emitter 104 described above with reference to previous Figures. In certain embodiments, a deodorizer chemical source 106 stores a deodorizer chemical. In FIG. 4, the deodorizer chemical is a liquid (specifically, an aqueous solution of chlorine dioxide). In this depicted embodiment, the deodorizer chemical source 106a is a liquid reservoir and the emitter 104a is an atomizer to emit aerosolized droplets of the aqueous chlorine dioxide solution. In FIG. 5, the deodorizer chemical is a gas (specifically, pressurized ozone gas). In this depicted embodiment, the deodorizer chemical source 106b is a pressurized gas cartridge and the emitter 104b is a valve for releasing the ozone into the tube 102.

In some embodiments, the deodorizer chemical source 106 provides one or more precursors to the emitter 104, and the emitter 104 produces the deodorizer chemical from the one or more precursor chemicals. In FIG. 6, the deodorizer chemical source 106c stores and provides (diatomic) oxygen gas as a precursor to ozone, and the emitter 104c produces the ozone gas. In some embodiments, the deodorizer chemical source 106 produces the deodorizer chemical from one or more precursor chemicals. In FIG. 7, the deodorizer chemical source 106d stores sodium chlorite and hydrochloric acid separately as precursor chemicals to chlorine dioxide, and permits the precursor chemicals to mix to deliver chlorine dioxide to the emitter 104d when the emitter 104d is operated.

Examples of storing or producing deodorizer chemicals are disclosed herein for illustrative purposes and are not intended as limiting. Various other or further deodorizer chemicals or chemical reactions for producing deodorizer chemicals, may be used in an apparatus for deodorizing exhaled smoke or vapor.

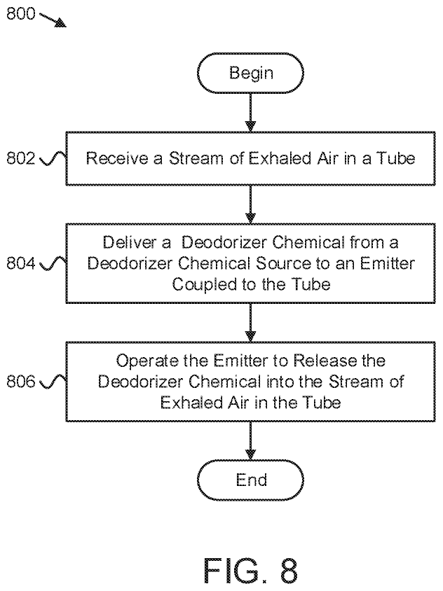

FIG. 8 is a schematic flow chart diagram illustrating one embodiment of a method 800 for deodorizing exhaled smoke or vapor. The method 800 begins, and a stream of exhaled air is received 802 in a tube 102. A deodorizer chemical source 106 delivers 804 a deodorizer chemical to an emitter 104 coupled to the tube 102. The emitter 104 is operated 806 to release the deodorizer chemical into the stream of exhaled air in the tube 102, and the method 800 ends.

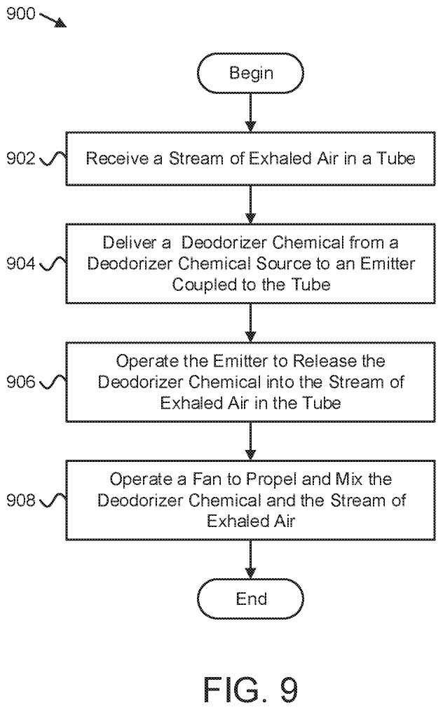

FIG. 9 is a schematic flow chart diagram illustrating another embodiment of a method 900 for deodorizing exhaled smoke or vapor. The method 900 begins, and a stream of exhaled air is received 902 in a tube 102. A deodorizer chemical source 106 delivers 904 a deodorizer chemical to an emitter 104 coupled to the tube 102. The emitter 104 is operated 906 to release the deodorizer chemical into the stream of exhaled air in the tube 102. A fan 304 disposed in the tube 102 is operated 908 to propel the stream of exhaled air past one or more baffles 308 to mix the deodorizer chemical with the stream of exhaled air before the stream of exhaled air exits the tube 102, and the method 900 ends.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An apparatus comprising:
a tube for receiving a stream of exhaled air;
an emitter coupled to the tube, the emitter operable to emit a deodorizer chemical into the stream of exhaled air in the tube; and
a deodorizer chemical source coupled to the emitter for delivering the deodorizer chemical to the emitter.

2. The apparatus of claim 1 further comprising one or more baffles disposed in the tube to mix the deodorizer chemical with the stream of exhaled air before the stream of exhaled air exits the tube.

3. The apparatus of claim 1, wherein the deodorizer chemical comprises an oxidizing agent.

4. The apparatus of claim 3, wherein the oxidizing agent is chlorine dioxide.

5. The apparatus of claim 3, wherein the oxidizing agent is ozone.

6. The apparatus of claim 1, further comprising:
a mouthpiece coupled to an intake end portion of the tube, for receiving the stream of exhaled air from a user;
a fan disposed in the tube and operable with the emitter to propel the stream of exhaled air toward an exhaust end portion of the tube; and
an air intake opening coupled to the intake end portion, the air intake opening permitting the tube to receive a secondary airflow in addition to the stream of exhaled air.

7. The apparatus of claim 6, further comprising:
a receptacle for holding an ingredient to be atomized and inhaled; and
an atomizer coupled to the receptacle and the tube, the atomizer operable to atomize the ingredient for inhalation through the mouthpiece.

8. The apparatus of claim 7, further comprising an airflow-operated switch that operates the emitter in response to a user exhaling through the mouthpiece and that operates the atomizer in response to the user inhaling through the mouthpiece.

9. The apparatus of claim 1, further comprising a switch operable by a user to operate the emitter.

10. The apparatus of claim 1, further comprising a sensor disposed in the tube, the sensor configured to produce an emitter activation signal in response to sensing the stream of exhaled air.

11. The apparatus of claim 10, further comprising control circuitry configured to operate the emitter in response to the emitter activation signal.

12. The apparatus of claim 1, wherein the deodorizer chemical source is a pressurized gas cartridge and the emitter is a valve.

13. The apparatus of claim 1, wherein the deodorizer chemical source is a liquid reservoir and the emitter is an atomizer.

14. The apparatus of claim 1, wherein the deodorizer chemical source stores the deodorizer chemical.

15. The apparatus of claim 1, wherein the deodorizer chemical source produces the deodorizer chemical from one or more precursor chemicals.

16. The apparatus of claim 1, wherein the deodorizer chemical source provides one or more precursors to the emitter, and the emitter produces the deodorizer chemical from the one or more precursor chemicals.

17. The apparatus of claim 1, wherein the deodorizer chemical source is a replaceable cartridge.

18. The apparatus of claim 17, further comprising a cartridge level indicator.

19. A method comprising
receiving a stream of exhaled air in a tube;
delivering a deodorizer chemical from a deodorizer chemical source to an emitter coupled to the tube; and
operating the emitter to release the deodorizer chemical into the stream of exhaled air in the tube.

20. The method of claim 19, further comprising operating a fan disposed in the tube to propel the stream of exhaled air past one or more baffles to mix the deodorizer chemical with the stream of exhaled air before the stream of exhaled air exits the tube.
